(19) United States
(12) Patent Application Publication
(10) Pub. No.: US 2015/0125917 A1
(43) Pub. Date: May 7, 2015

(54) D(-) LACTIC ACID PRODUCTION

(71) Applicant: Cellulac Limited, Parkmore, Galway (IE)

(72) Inventors: Patrick Walsh, Galway (IE); Joachim Venus, Potsdam (DE)

(21) Appl. No.: 14/397,619

(22) PCT Filed: May 2, 2013
(86) PCT No.: PCT/EP2013/059186

§ 371 Date: Oct. 28, 2014

(30) Foreign Application Priority Data
May 2, 2012 (IE) S2012/0220

(51) Int. Cl. C12P 7/56

(52) U.S. Cl. CPC C12P 7/56; C12P 2203/00; C12P 2201/00
USPC 435/139

(57) ABSTRACT

A process for producing D(-) lactic acid in high enatiopurity from lignocellulosic material includes providing a hydrolysate of cellulose polymers prepared from the lignocellulosic material, which includes hexose and pentose sugars and contacting the hydrolysate with the bacterium Lactobacillus coryniformis subsp. Torquens, strain 30 (ATCC 25600) in a fermentation reaction.

This invention relates to the conversion of lignocellulosic biomass materials into lactic acid in high yield.

The industrial production of fermentation products such as ethanol and lactic acid, is facing the challenge of redirecting the production process from fermentation of relatively easily convertible but expensive starchy materials, to the complex but inexpensive lignocellulosic biomass such as wood and residues from agricultural crops, e.g. straw. Unlike starch, which contains homogenous and easily hydrolysed polymers, lignocellulosic biomass contains cellulose, hemicellulose, polyphenolic lignin and other extractable components. Lignocellulosic biomass is the most abundant renewable material on the planet and has long been recognised as a potential feedstock for producing chemicals, fuels and other materials. As used herein, lignocellulosic biomass means any material containing cellulose, any cellulose and lignin. Examples of such materials include but are not limited to corn strover, corn fibre, wheat straw, rice straw, sugarcane bagasse, hardwoods, softwoods, pulp and paper wastes, recycled paper, forest residues and process streams containing of these materials.

Cellulose, which is a B-glucan built up of anhydro D-glucose units, is the main structural component of plant cell walls and normally constitutes about 30-60% by weight of lignocellulosic materials. Hemicellulose, is a term utilised to denote non-cellulosic polysaccharides associated with cellulose in plant tissues. Hemicellulose frequently constitutes 20-35% w/w of lignocellulosic materials. Lignin is a complex cross linked polymer based on variously substituted p-hydroxyphenylpropane units and generally constitutes about 10-30% w/w of lignocellulosic materials.

There are two optical isomers of lactic acid: L(+) lactic acid and D(-) lactic acid. While pure optical isomers of lactic acid can be obtained by microbial fermentation, racemic lactic acid is always produced via chemical synthesis. In order to achieve the desired polymer properties, high purity D- or L+ lactic acid monomers are necessary.

Typically, the first step in utilization of lignocellulosic biomass in production of lactic acid is a pre-treatment process, in order to fractionate the components of lignocellulosic material and increase their surface area. The pre-treatment method most often used is acid hydrolysis, where the lignocellulosic material is subjected to an acid such as sulphuric acid whereby the sugar polymers cellulose and hemicellulose are partly or completely hydrolysed to their constituent sugar monomers. Another type of lignocellulose hydrolysis is steam explosion, a process comprising of heating the lignocellulosic material by steam injection to a temperature of 190-230° C. A third method is wet oxidation wherein the material is treated with oxygen at 150-185° C. The pre-treatments can be followed by enzymatic hydrolysis to complete the release of sugar monomers. These pre-treatment steps result in the hydrolysis of cellulose into the hexoses, glucose and galactose while hemicellulose is transformed into the pentoses, xylose and arabinose. Thus, in contrast to starch, the hydrolysis of lignocellulosic biomass results in the release of pentose sugars in addition to hexose sugars. This implies that useful fermenting organisms need to be able to convert both hexose and pentose sugars to desired fermentation products such as lactic acid. Traditional microorganisms used do not efficiently metabolize pentoses such as xylose and arabinose, and extensive metabolic engineering is thus necessary to improve performance on lignocellulosic substrates.

Lignocellulose hydrolysates may contain inhibitors such as furfural, phenols and carboxylic acids, which can potentially inhibit the fermenting organism. Therefore, the organism must also be tolerant to these inhibitors. The inhibitory effect of the hydrolysates can be reduced by applying a detoxification process prior to fermentation. However, the inclusion of this extra process step increases significantly the total cost of the fermentation product. Therefore it would be economically beneficial that the microorganisms used are capable of producing fermentation products from undetoxified hemicellulose or cellulose hydrolysates to make them usable in an industrial lignocellulosic-based fermentation process, due to the high cost of detoxification process.

Lactic acid is widely used in the food, pharmaceutical and textile industries. It is also used as a source of lactic acid polymers which are used in degradable plastics. Optically pure lactic acid is currently produced, for example, by the fermentation of glucose derived from cornstarch using various lactic acid bacteria. Lactobacillus species are used extensively in industry for starch based lactic acid production, the majority of which lack the ability to efficiently ferment pentose sugars such as xylose and arabinose as well as the hexose sugars such as glucose. Although certain lactobacillus species can additionally ferment pentose sugars to lactic acid, pentoses are metabolised using the phosphoketolase pathway which is inefficient for lactic acid production because acetic acid and/or ethanol is produced along with the lactic acid. The present inventors have now identified a particular strain of lactobacillus species that, advantageously, may be used in the production of lactic acid and which has the ability to efficiently metabolise pentose and hexose sugars to produce high yields of optically pure D (-) lactic acid.

It is a goal of the present invention to provide a process that overcomes some of the above mentioned problems.

### SUMMARY OF THE INVENTION

In accordance with a first aspect of the present invention, there is provided a process for producing D(-) lactic acid in high enatiopurity from lignocellulosic material, the process comprising, providing a hydrolysate of cellulose polymers prepared from said lignocellulosic material and comprising hexose and pentose sugars, contacting said hydrolysate with the bacterium Lactobacillus coryniformis subsp. Torquens, strain 30 (ATCC 25600) in a fermentation reaction.

Advantageously, the bacterium has been found to metabolise hexose and pentose sugars with high efficiency to produce entiomerically very pure D(-) lactic acid. In one embodiment, the process comprises the steps of:
a steam explosion pre-treatment step to fractionate the lignocellulosic material into lignin, cellulose and hemicellulosic fractions;
an enzymatic treatment step to hydrolyse cellulose polymers to hexose and pentose sugars; and,
a lactic acid production step comprising producing lactic acid from the hexose and pentose sugars, wherein, the hexose and pentose sugars are converted by strain designation 30 of Lactobacillus coryniformis subsp. torquens (ATCC 25600) in a fermentation reaction.

The process optimises the production of highly enantiomerically pure lactic acid in conjunction with the highly selected strain of lactic acid bacteria and is designed specifically for this purpose. No system disclosed to date, meets all of the requirements for such high yields and efficient production.

Lactic acid production systems using lignocellulosic biomass as the source material generally have to undergo a pre-treatment process to separate the lignin, cellulose and hemicellulosic fractions. Many appropriate pre-treatment processes can be used. A physical pre-treatment can, for example, be achieved by grinding the lignocellulosic material. Alternatively a chemical process can be used where dilute acid is added. Furthermore, a physiochemical process such as steam treatment can be used.

In one embodiment of the present invention, a steam explosion pre-treatment process is used to fractionate the components of lignocellulosic material. The steam explosion pre-treatment process comprises the following steps:
Lignocellulosic material is reduced in size to 2 to 3 cm and pre-soaked at 60° C.;
The reduced and pre-soaked lignocellulosic material is moved into the digester and held at approximately 15 bar pressure and approximately 200° C. for approximately 2 to 3 minutes;
The material is explosively released through a blow valve into an expansion tank.

The steam explosion pre-treatment process further opens the cell wells of the lignocellulosic material and separates the lignin, cellulose and hemicellulosic fractions. The process also exposes the cellulose polymers, so that enzymes can penetrate and hydrolyse the polymers to C6 monomers although some C6 monomers will be produced in the steam explosion step. These C6 monomers are then ready for fermentation to D(-) lactic acid by strain designation 30 of Lactobacillus coryniformis subsp. torquens.

Advantageously, it has been found that utilising the steam explosion pre-treatment process using a steam explosion system produces a fraction that contains less enzyme inhibitors than other available systems thereby increasing even further the potential of the system. The essential parameters for steam explosion systems, is that they produce material that have very low levels of ‘inhibitors’. Inhibitors are products chemically produced during the steam and pressure treatment if the residence time is incorrect. If the level of inhibitors are too high in the hydrolysate (the material is referred to as hydrolysate after being fractionated in the steam and pressure system and explosively released into a flash tank) the enzymes and bacteria used in the rest of the process will denature or die, resulting in complete or partial collapse of the process.

Steam explosion involves heating the biomass using high-pressure saturated steam (at approximately 200° C.) for a short period (approximately 2 to 3 minutes). Steam condenses under high-pressure, thereby wetting the material and then the pressure is suddenly reduced which makes the material undergo an explosive decompression.

The method of the present invention further comprises the step of enzymatic hydrolysis of the cellulose and hemicelluloses, which are produced during the steam explosion pre-treatment process. In the method of the present invention, the enzyme hydrolysis step is carried out using cellulases commercially available under the trade name CellicCTec2 of CellicHTec2 from Novozymes. Alternatively, the enzyme hydrolysis step may be carried out using cellulases commercially available under the trade name Accellerase DUET from Genencor. Similarly, enzymes may be produced from the bacterial strain Trichoderma reesei and utilised in the enzyme hydrolysis step.

Preferably, the enzymes are added at a rate of 6 ml enzymes per kg of pre-treated material at approximately 20% moisture content. Nutrient supplements such as, for example yeast, grass juice, waste streams from dairy production, corn steep liquor and peptanes may be added at an appropriate rate to such as 15 g/litre of yeast extract.

Levels of monosaccharides, di- and oligosaccharides, furfural, hydroxymethylfurfuran and furfuryl alcohol in the hydrosylate are analyzed and quantified by HPLC using an Altech IOA-1000 column at 90° C., with 3 mM sulfuric acid as the mobile phase (0.4 ml per min), followed by detection by differential refractometry.

As aforementioned, generally different microbial systems are required to efficiently convert both hexose and pentose sugars to lactic acid and the identification of Lactobacillus strains capable of metabolising both types of sugars offers significant advantages in terms of the design and production of systems that are capable of efficiently producing lactic acid.

Advantageously, Lactobacillus strain designation 30 of Lactobacillus coryniformis subsp. Torquens, a homofermentative D(-) lactic acid producing strain, has been found to convert in excess of 65% of all of the sugars to D(-) lactic acid at an enantiomeric purity greater than 99%.

Generally, it is necessary to separate the pentose and hexose sugars immediately after a steam treatment process in a detoxification step, to produce a ‘higher purity’ medium for the bacteria to survive. The strain used in accordance with the present invention can advantageously survive in the medium produced following the lignocellulosic pre-treatment step. Therefore, the bacterial strain is capable of surviving and reproducing in the produced medium of the sugars, lignin, proteins, fatty acids, low levels of furans and 2-hydroxymethylfurfural and which avoids the need for an additional and expensive process step to separate out the sugars before enzymatic hydrolysis and fermentation.

Therefore, the method according to the invention specifically embraces the step of adding the bacterial strain directly to the hydrolysate obtained from a pre-treatment step carried out prior to enzyme hydrolysis of the cellulose and hemicellulose fractions into their component sugars.

During the fermentation reaction, the pH is balanced within the range of 5.5 to 6.5. However, preferably the pH of the fermentation reaction is maintained at approximately 6.0. Preferably, for D(-) producing homofermentative bacterial strain designation 30 of Lactobacillus coryniformis subsp. Torquens, the temperature is maintained in the range of 28 to 32° C. but preferably at approximately 30° C.

The fermentation may be a continuous process rather than a batch process, which would help to improve process profitability.

The process of the present invention also includes the additional steps of further Downstream Processing (DSP) where the lactic acid is separated from the other materials and is present as sodium lactate. Microfiltration allows separation of the bacterial cells in the medium. These bacterial cells may than advantageously be recycled back into the fermenting tank to reduce the cost of nutrients and increase efficiency for a continuous production of lactic acid as opposed to batch production.

The lignin is separated and stored. Lignin, when burned has an energy content of ˜27 MJ/kg compared ˜17 MJ/kg for woodchips or pellets. The lignin may advantageously be sold as a high energy pellet for domestic pellet burners or burned on site to produce energy for the system.

The remaining material may then be passed through a softening and ultrafiltration system to separate the sodium lactate from the retentate. Sodium (Na) may then be separated from the lactate during for example bipolar electrodialysis. Sodium Hydroxide (NaOH) may be reformed and recycled into the enzymatic hydrolysis and the bacterial fermenter to reduce cost of chemicals.

The remaining material has approximately 20% volatile solids. This material may be piped to an anaerobic digester for example where methane (CH4) is generated. The methane is combusted in a combined heat and power unit (CHP) where heat (steam) and electricity is produced to run the plant. Estimated production of heat and electricity means the plant would be self-sufficient in energy resulting in significant savings during the production process.

All water is treated and recycled into the system resulting in low water demand after the initial start-up.

### BRIEF DESCRIPTION OF THE DRAWINGS

The invention will be more clearly understood from the following description of some embodiments thereof, given by way of example only, with reference to the accompanying drawings, in which:

FIG. 1 is a schematic overview of the a method according to a first embodiment of the present invention;

FIG. 2 is a graphic representation of the results obtained for C5 and C6 sugar conversion using Lactobacillus strain designation 30 of Lactobacillus coryniformis subsp. torquens in accordance with the method of the present invention;

FIG. 3 is a graphic representation of the results obtained following downstream processing of the lactic acid produced in the method of the invention;

FIG. 4 is a comparison between performance of bacteria at two different levels of production.

### DETAILED DESCRIPTION OF PREFERRED EMBODIMENTS

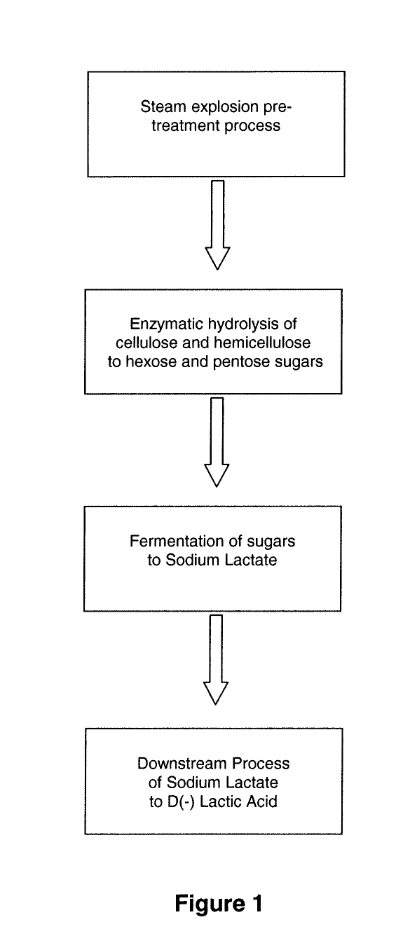

Referring to FIG. 1, there is provided a schematic overview of a method according to a first embodiment of the present invention. The method comprises 3 steps; a steam explosion pre-treatment process, an enzymatic hydrolysis step and a fermentation step. The enzymatic hydrolysis step comprises hydrolysis of cellulose and hemicelluloses to pentose and hexose sugars. The fermentation step comprises the addition of Lactobacillus strain designation 30 of Lactobacillus coryniformis subsp. Torquens to the hydrosylate to result in the production of D(-) lactic acid.

The following is an example of a method according to the invention:

1. Lignocellulosic material is placed under pressure and high Temperature using steam in a proprietary ‘steam explosion’ system. Residence time is approximately 2 to 3 minutes at approximately 200° C. and 15 bar pressure.

2. The material is released explosively into an expansion chamber at which time the cellulose, hemicellulose and lignin are exposed from the cell structures.

3. The material is rehydrated/diluted with water (H2O) at a rate of 1.45 kg H2O/kg raw material.

4. pH is adjusted using sodium hydroxide (NaOH) to minimum pH 5 max pH 6. The Temperature is maintained at 52° C.

5. Cellulase enzymes e.g. Cellic Ctec2 (Novozymes) are added at a rate of 6 ml enzymes per kg pretreated material at 20% moisture content.

6. The hydrolysed material is moved to a fermenter where bacterial cultures are added.

7. Nutrient supplements yeast and peptanes are added at a rate of 15 g/litre yeast extract.

8. Utilising Lactobacillus strain designation 30 of Lactobacillus coryniformis subsp. torquens maintained at a temperature of 30° C. and constant automated adjustment of pH by NaOH, D(-) lactic acid is produced.

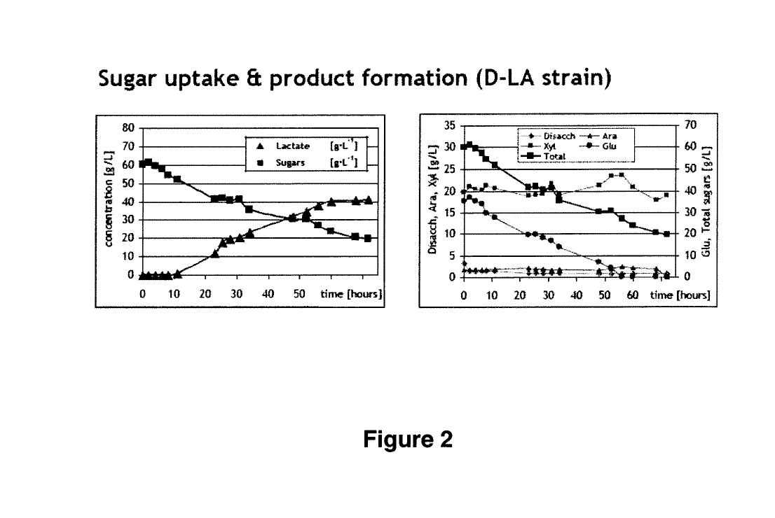

FIG. 2 shows a graphic representation of the results obtained for C5 and C6 sugar conversion using Lactobacillus strain 30 of Lactobacillus coryniformis subsp. torquens. This strain of bacteria can convert 65% of lignocellulosic sugars to D(-) lactic acid at high enantiopurity of >99%. Complete (100%) conversion of glucose to D(-) lactic acid is also indicated as a control.

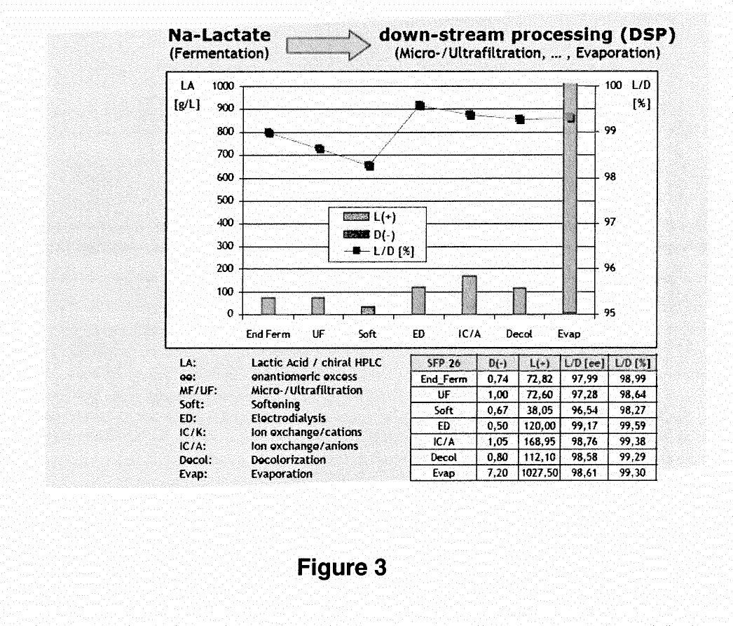

Referring now to FIG. 3, there is provided a graphic representation of the results obtained following downstream processing of the lactic acid produced in the method of the invention. By using a downstream processing system of ultrafiltration, softening, electrodialysis, ion exhange, decolourization and evaporation, several grades of D(-) lactic acid, up to pharmaceutical grade lactic acid, can be produced.

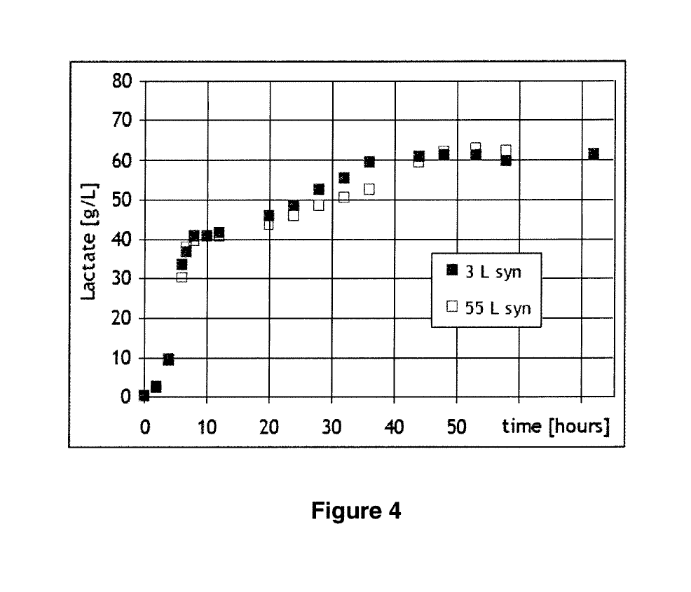

FIG. 4 shows a graphic representation of a comparison between performance of bacteria at two different levels of production. No difference was found between the two production levels in yields and efficiency.

The terms “comprise” and “include”, and any variations thereof required for grammatical reasons, are to be considered as interchangeable and accorded the widest possible interpretation.

The invention is not limited to the embodiments hereinbefore described which may be varied in both construction and detail within the scope of the appended claims.

1. A process for producing D(-) lactic acid in high enatiopurity from lignocellulosic material, the process comprising, providing a hydrolysate of cellulose polymers prepared from said lignocellulosic material and comprising hexose and pentose sugars, contacting said hydrolysate with the bacterium Lactobacillus coryniformis subsp. Torquens, strain 30 (ATCC 25600) in a fermentation reaction.

2. A process according to claim 1 wherein a steam explosion pre-treatment process is used to fractionate the components of the lignocellulosic material.

3. A process according to claim 2 wherein said steam explosion pre-treatment step utilises a steam explosion system.

4. A process according to claim 2 wherein the steam explosion pre-treatment step comprises heating the lignocellulosic biomass with steam under pressure of approximately 15 bar for a period of approximately 2 to 3 minutes at a temperature of approximately 200 degrees centigrade.

5. A process according to claim 2 further comprising an enzyme hydrolysis step wherein cellulase enzymes are added to the lignocellulosic material following the steam explosion pre-treatment step.

6. A process according to claim 5 wherein the cellulases comprise any of the cellulases commercially available under the trade name CellicCTec2 of CellicHTec2 or under the trade name Accellerase DUET, or cellulose enzymes isolated from the bacterial strain Trichoderma reesei.

7. A process according to claim 5, wherein said enzymes are added at a rate of 6 ml enzymes per kg of pre-treated material at approximately 20% moisture content.

8. A process according to claim 5 further comprising adding nutrient supplements to the lignocellulosic material.

9. A process according to claim 1 wherein the pH of the fermentation reaction is maintained at 5.5 to 6.5.

10. A process according to claim 1 wherein the temperature of the fermentation reaction is maintained at 28 to 32° C.

11. A process according to claim 1 wherein the fermentation reaction is a continuous process.

12. A process according to claim 1 wherein the lactic acid produced is separated from the hydrolysate after fermentation.

13. A process according to claim 1 wherein the bacterial cells are filtered from the hydrolysate and fed back to the reaction.

14. A process according to claim 1 wherein the lignin is separated from the hydrolysate.

15. A process for producing D(-) lactic acid in high enatiopurity from lignocellulosic material comprising contacting the material with an amount of the bacterium Lactobacillus coryniformis subsp. Torquens, strain 30 (ATCC 25600), which is effective to produce D(-) lactic acid in high enantiopurity.

16. A process according to claim 8, wherein the nutrient supplements are selected from the group consisting of yeast, grass juice, waste streams from dairy production, corn steep liquor, peptanes and combinations thereof.

17. A process according to claim 9, wherein the pH of the fermentation reaction is maintained at 6.0.

18. A process according to claim 10, wherein the temperature of the fermentation reaction is maintained at 30° C.
